(19) United States
(12) Patent Application Publication
(10) Pub. No.: US 2022/0023497 A1
(43) Pub. Date: Jan. 27, 2022

(54) AMINO ACID ENRICHED TUNABLE BIOINK FORMULATION FOR MULTIDIMENSIONAL BIOPRINTING AND THE PROCESS THEREOF

(71) Applicant: SREE CHITRA TIRUNAL INSTITUTE FOR MEDICAL SCIENCES AND TECHNOLOGY, THIRUVANANTHAPURAM (IN)

(72) Inventors: Roy JOSEPH, THIRUVANANTHAPURAM (IN); Praveen Kunjan SOBHAN, THIRUVANANTHAPURAM (IN)

(73) Assignee: SREE CHITRA TIRUNAL INSTITUTE FOR MEDICAL SCIENCES AND TECHNOLOGY, THIRUVANANTHAPURAM (IN)

(21) Appl. No.: 17/311,879

(22) PCT Filed: Jan. 10, 2020
(86) PCT No.: PCT/IN2020/050026

§ 371 Date: Jun. 8, 2021

(30) Foreign Application Priority Data
Jan. 11, 2019 (IN) 201941001306

(51) Int. Cl. A61L 27/22; B33Y 70/00; A61L 27/38; A61L 27/54; A61L 27/02; B33Y 10/00

(52) U.S. Cl. CPC A61L 27/222; B33Y 70/00; A61L 27/38; A61L 2400/18; A61L 27/025; B33Y 10/00; A61L 27/54

(57) ABSTRACT

A 3D bioprintable ink formulation capable of multidimensional printing comprises gelatin modified by an essential amino acid arginine, oxidized alginate and a catalyst.

### FIELD OF THE INVENTION

The present invention relates to a novel bioresorbable ink formulation or more particularly a 3D bioprintable hydrogel with modified version of gelatin crosslinked with oxidized alginate.

### BACKGROUND OF THE INVENTION

One of the most important components of 3D bio-printing is the bioink or the biologically favorable material formulations, which is a mixture of cells, biomaterials and bioactive molecules that creates the printed structures. Bioink or the biomaterial formulation is the critical component of 3D bioprinting. The production of bioinks with diverse chemistries, compositions, mechanical characteristics, biological properties, and degradation kinetics will advance the bioprinting field forward toward application in many tissues and organs. Currently available bioinks involve a wide range of biomaterials and their blends. With the advancement of 3D bioprinting, the search for biologically inspired physiologically relevant bioink formulations has emerged. This is very important in maintaining and sustaining the viability, proliferation, maintenance and functionality of various cell types.

Indian patents No. 235249 and 214429 deals with process and application of modified alginate cross-linked with gelatin exclusively for the preparation of a biopolymer matrix. It does not include or indicate preparation and process for Bioink. Indian patent application No 201841020267 does not have amino acid enrichment. Patent Number WO2015173020 deals with bone implants only and do not have the tunable property and do not have any bioavailability of essential amino acids required for physiological requirements. Patent Number WO2017214592 does not have the tunable property and do not have any bioavailability of essential amino acids required for physiologica requirements. Patent application number PCT/US2017/035861 does not have the tunable property and do not have any bioavailability of essential amino acids required for physiological requirements. Alginate is one of the most commonly used materials in bioinks (e.g., ‘CELLINK A’ by M/s. CELLINK) being utilized in the bioprinting field, being utilized as a standalone bioink and as a base material for composite bioinks. But the tunability of alginate based bioinks in terms of gelation time, viscosity, elasticity, compression and its degradation time is poor and do not have any bioavailability of essential amino acids required for physiological requirements.

But the conventional bioinks often lacks in maintaining and sustaining the viability, proliferation, maintenance and functionality of various cell types.

Further, with the emergence and applicability of 3D bioprinting in regenerative medicine, prosthetics, implants, disease biology studies, toxicology and drug screening, the demand for robust and mechanically tunable bioink is growing. Global 3D bioprinting market is expected to reach USD2.6 billion by 2024.

The present invention address this issue by providing a 3D bioprintable arginine bonded gelatin-oxidized alginate formulation, as the formulation is tunable in terms of gelation time, viscosity, elasticity, resistance to deformation under low compression load and its degradation time and have the bioavailability of essential amino acid required for physiological requirements.

### OBJECTS OF THE INVENTION

It is therefore the principle object of the present invention is to provide a 3D bioprintable hydrogel based ink formulation comprised of modified version of gelatin with oxidized alginate and catalyst.

Another object of the present invention is to provide 3D bioprintable ink, which provides the advantage of shear thinning property of gelatin and the versatility of alginate.

Yet another object of the present invention is to provide 3D bioprintable ink, which is robust and mechanically tunable.

Further object of the present invention is to provide 3D bioprintable ink, which can be useful in regenerative medicine, prosthetics, implants, disease biology studies, toxicology and drug screening.

Another object of the present invention is to provide 3D bioprintable ink, which is ready to use and ready to print in various media.

Yet another object of the present invention to provide a method of producing 3D bioprintable ink, which does not require any mutagenic source/chemicals such as UV ray or any harmful thermal cross linking agent.

### SUMMARY OF THE INVENTION

One or more drawbacks of conventional bioresorbable ink formulation is overcome, and additional advantages are provided through the composition as claimed in the present disclosure. Additional features and advantages are realized through the technicalities of the present disclosure. Other embodiments and aspects of the disclosure are described in details herein and are considered to be part of the claimed disclosure.

A 3D bioprintable ink formulation capable of multidimensional printing comprises gelatin modified by an essential amino acid arginine, oxidized alginate and a catalyst.

Various objects, features, aspects, and advantages of the inventive subject matter will become more apparent from the following detailed description of preferred embodiments.

It is to be understood that the aspects and embodiments of the disclosure described above may be used in any combination with each other. Several of the aspects and embodiments may be combined to form a further embodiment of the disclosure.

The foregoing summary is illustrative only and is not intended to be in any way limiting. In addition to the illustrative aspects, embodiments, and features described above, further aspects, embodiments, and features will become apparent by reference to the drawings and the following detailed description.

### DETAILED DESCRIPTION OF THE PRESENT INVENTION

While the embodiments of the disclosure are subject to various modifications and alternative forms, specific embodiment thereof have been shown by way the figures and will be described below. It should be understood, however, that it is not intended to limit the disclosure to the particular forms disclosed, but on the contrary, the disclosure is to cover all modifications, equivalents and alternative falling within the scope of the disclosure.

It is to be noted that a person skilled in the art would be motivated from the present disclosure to arrive at a bioresorbable ink formulation and method of preparing the same. Such method for evaluating the same may vary based on combination of one or more ingredients. However, such modifications should be construed within the scope of the disclosure. Accordingly, the drawings illustrate only those specific details that are pertinent to understand the embodiments of the present disclosure, so as not to obscure the disclosure with details that will be clear to those of ordinary skill in the art having benefit of the description herein.

As used in the description herein and throughout the claims that follow, the meaning of “a”, “an”, and “the” includes plural reference unless the context clearly dictates otherwise. Also, as used in the description herein, the meaning of “in” includes “in” and “on” unless the context clearly dictates otherwise.

The terms “comprises”, “comprising”, or any other variations thereof used in the disclosure, are intended to cover a non-exclusive inclusion, such that a method, composition coating, electrode, toughness that comprises a list of components does not include only those components but may include other components not expressly listed or inherent to such method, or assembly, or method. In other words, one or more elements in a system or device proceeded by “comprises . . . a “does not, without more constraints, preclude the existence of other elements or additional elements in the system, apparatus or device.

The present invention relates to a bioresorbable ink formulation to be used in 3D bioprinting comprises 3D bioprintable hydrogel with modified version of gelatin containing arginine crosslinked with oxidized alginate in presence of small concentration of catalyst having suitable mechanical properties of 3D bioink material. This formulation can be used as physiologically relevant and biologically inspired bioink for 3D bioprinting.

This formulation can be used to print hepatocytes, liver cells, brain cells, skin cells, cartilage, bone, skeletal and cardiac muscles, lung, pancreatic, kidney, intestine, stem cells, mesenchymal stem cells and induced pluripotent stem cells (iPSC) derived cells. The printing of these cells with the material formulation will not undermine the functionality and longevity of these cells, thus maintaining similar properties in normal in vivo physiological conditions.

Thus this formulation can be used for 3D bioprinting and subsequent development of: physiologically similar systems, surgical implants, toxicological screening systems, drug screening assay systems, metabolic study and screening systems, disease biology model systems, developing regenerative systems, developing various artificial cell types, tissues, organoids and organs.

The catalyst used in this formulation is minerals or modified mineral such as borax.

The arginine modified gelatin and oxidized alginate are taking in the range of 1% to 30% and was mixed in the ratio of 1:1 to 1:20 or vice versa. The concentration of catalyst such as borax is ranging from 0.1% to 10%.

The bioprintable ink formulation can optionally be conjugated or modified with growth factors, proteins, nutrients, minerals, vitamin, iron, peptides, antibiotics, adjuvant, amino acids, growth enhancers for multidimensional bioprinting.

In accordance with another embodiment of the present invention, the oxidation process of sodium alginate and modification of gelatin were also provided.

### Oxidation of Sodium Alginate

1 to 30 g sodium alginate was dispersed in 1-200 mL ethanol and sodium metaperiodate in 1-200 mL distilled water was added to it along with magnetic stirring in dark for 6-12 hours to obtain the oxidized alginate of different degree of oxidation.

The degree of oxidation was followed by determining the concentration of periodate left unconsumed by iodometry after 6-12 h. A 1-15 mL aliquot of the reaction mixture was neutralized with 1-50 mL of 1-30% sodium bicarbonate solution. Iodine was liberated by the addition of 1-50% potassium iodide solution. This was kept under dark for 1-50 min and liberated iodine was then titrated with standardized sodium thiosulphate solution using starch as the indicator.

After the reaction, the solution was dialysed against distilled water (1-50 L) for 1-70 hour till the dialyzate was periodate free and then the dialyzate was then freeze dried. The yield of the oxidized products ranged from 5% to 90%.

### Modification of Gelatin using Arginine

The carboxyl group in gelatin was modified when in treatment with an amino acid arginine. The strong Hydrogen bond between amino group and carboxyl groups gets disrupted and as a result, gelatin solution could maintain its liquid state at room temperature.

At first 1-20 g gelatin was dissolved in 1-1000 mL of distilled water and 1-30 g of L-Arginine was added to it along with adjustment of pH in the range of 1-5 using HCl.

1-100 g of 1-Ethyl-3-(3-dimethylaminopropyl) carbodiimide hydrochloride (EDC) was then added to the solution and the reaction was allowed to take place for 1-100 hours at 1-37° C. range with continuous stirring at 1-2000 rpm. After the completion of the reaction, the reaction mixture was transferred to 1-35 KDa dialysis membrane and the dialysis was continued for 1-30 days with double exchange of water each day. After the dialysis, the solution was freeze dried and the obtained modified gelatin was weighed and packed.

In accordance with another embodiment of the present invention, there is provided a technique for 3D bioprinting with gel forming composition.

The 3D bioprintable formulation was first dissolved in phosphate buffered Saline (PBS) to turn the solution mixture into a gel. Apart from PBS, other cell surviving media can also be used as well. Gelation time can also be varied with varying concentration of oxidized alginate, gelatin and borax.

Post-printing gelation time can be adjusted from 1 minute to 120 minutes by varying solution concentration of oxidized alginate and gelatin in the range 1% to 30% and by changing the mixing ratio 1:1 to 1:20 and vice versa.

The gelation rate can be further tuned by catalyzing the reaction by adding minerals or modified minerals such as borax having concentrations ranging from 0.1% to 10%.

With the help of the 3D bioprintable formulation, twenty layers printing cell can be found out.

It was also possible to print gels loaded with various cell types. The printing did not found to alter viability, proliferation, functionality and metabolic maintenance of cell types tested.

The bioprintable ink formulation can be conjugated or modified with growth factors, proteins, nutrients, minerals, vitamins, ions, peptides, antibiotics, adjuvant, amino acids and growth enhancers.

The bioprintable ink can be used for multidimensional bioprinting with various cell types but not limited to human or animal hepatocytes, liver cells, skin cells, brain cells, cartilage, bone, endothelial cells, blood cells, skeletal and cardiac muscles, lung, pancreatic, kidney, intestine, stem cells, mesenchymal stem cells and iPSC derived cells. The multidimensional bioprintable bioink is suitable for bioprinting of physiologically similar systems, toxicological screening systems, drug screening assay systems, metabolic study and screening systems, disease biology model system, developing medical regenerative systems, surgical implants, developing various artificial cell types, tissues, organoids and organs for transplant, in vitro, ex vivo and in vivo diagnostic and experimental model systems, regenerative medicines, prosthetics, cosmetics, implants, disease biology studies and toxicology and drug screening studies, artificial intelligence based medical regenerative treatments, implants, tools and procedures, augmented reality based medical regenerative treatments, implants, tools and procedures and block chain based medical regenerative treatments, implants, tools and procedures.

The printing of these cells with the material formulation will not compromise or undermine the functionality and longevity of these cells, thus maintaining similar properties as of these cells maintained at normal in vivo physiological conditions. Thus this formulation can be used for 3D bioprinting and subsequent development of physiologically similar systems, toxicological screening systems, drug screening assay systems, metabolic study and screening systems, disease biology model system, in vitro, ex vivo and in vivo diagnostic and experimental model systems developing regenerative systems, developing various artificial cell types, tissues, organoids and organs for transplant, artificial intelligence based medical regenerative treatments, implants, tools and procedures, augmented reality based medical regenerative treatments, implants, tools and procedures and block chain based medical regenerative treatments, implants, tools and procedures. The material is ready to use and ready to print in various culture media and do not have any mutagenic source in the formulation or does not have any harmful thermal crosslinking procedures.

Each of the appended claims defines a separate invention, which for infringement purposes is recognized as including equivalents to the various elements or limitations specified in the claims. Depending on the context, all references below to the “invention” may in some cases refer to certain specific embodiments only. In other cases, it will be recognized that references to the “invention” will refer to subject matter recited in one or more, but not necessarily all, of the claims.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all groups used in the appended claims.

The present disclosure provides a novel 3D bioprintable ink formulation and the methodology of preparing the same.

### Equivalents

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to”, the term “having” should be interpreted as “having at least”, the term “includes” should be interpreted as “includes but is not limited to”, etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/ or “an” should typically be interpreted to mean “at least one” or “one or more”); the same holds true for the use of definite articles used to introduce claim recitations. In addition, eve it a specific number of an introduced claim recitation is explicitly recited, those skilled in the art will recognize that such recitation should typically be interpreted to mean at least the recited number (e.g., the bare recitation of “two recitations”, without other modifiers, typically means at least two recitations, or two or more recitations).

The terminology used herein is for the purpose of describing particular embodiments only and is not intended to be limiting of the present disclosure. It will be appreciated that several of the above-disclosed and other features and functions, or alternatives thereof, may be combined into other systems or applications. Various presently unforeseen or unanticipated alternatives, modifications, variations, or improvements therein may subsequently be made by those skilled in the art without departing from the scope of the present disclosure as encompassed by the following claims.

The claims, as originally presented and as they may be amended, encompass variations, alternatives, modifications, improvements, equivalents, and substantial equivalents of the embodiments and teachings disclosed herein, including those that are presently unforeseen or unappreciated, and that, for example, may arise from applicants/patentees and others.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A 3D bioprintable ink formulation capable of multidimensional printing comprises gelatin modified by an essential amino acid such as arginine, oxidized alginate and a catalyst.

2. The 3D bioprintable ink formulation as claimed in claim 1, wherein the arginin modified gelatin and oxidized alginate are mixed in the ratio of 1:1 to 1:20 or vice versa.

3. The 3D bioprintable ink formulation as claimed in claim 1, wherein the arginine modified gelatin and oxidized alginate are provided in the range of 1% to 30%.

4. The 3D bioprintable ink formulation as claimed in claim 1, wherein the catalyst is the modified minerals such as borax.

5. The 3D bioprintable ink formulation as claimed in claim 1, wherein borax is used in the concentration of 0.1% to 10%.

6. The 3D bioprintable ink formulation as claimed in claim 1, further comprises growth factors, proteins, nutrients, minerals, vitamins, ions, peptides, antibiotics, adjuvant, amino acids, and growth enhancers.

7. A process of preparing the 3D bioprintable ink formulation comprises two steps.
i) oxidation of sodium alginate and
ii) modification of gelatin by arginine.

8. The process of preparing the ink formulation as claimed in claim 7, wherein the oxidation of sodium alginate comprises the steps of:
i) taking sodium alginate in ethanol and sodium metaperiodate mixed in distilled water was added to it along with magnetic stirring in the dark for 6 to 12 hours;
ii) checking of degree of oxidation by concentration of unconsumed periodate by iodometry after 6-12 hours;
iii) dialyzing of the solution against distilled water till the dialyzate was periodate free; and
iv) freeze drying of dialyzate.

9. The process of preparing the ink formulation as claimed in claim 7, wherein checking the degree of oxidation comprises the steps of:
i) neutralizing of 1 to 15 mL aliquot of reaction mixture with 1-30% sodium bicarbonate solution where iodine was liberated by addition of 1-50% potassium iodide solution;
ii) Keeping the reaction mixture under dark for 1-50 min., and titration of the liberated iodine with standardized sodium thiosulphate solution with starch as the indicator;

10. The process of preparing the ink formulation as claimed in claim 7, wherein the modification of gelatin comprises the steps of:
i) dissolving of 1-20 g gelatin in distilled water where L-arginine was added at a pH of 1-5 by using HCL;
ii) addition of 100 g of 1-Ethyl-3-(3-dimethylaminopropyl) carbodiimide hydrochloride to the mixture and the reaction takes place for 1-100 hours at 1-37° C. temperature with continuous stirring;
iii) transferring of reaction mixture to 1-35 KDa dialysis membrane and continued for 1-30 days; and
iv) freeze drying the solution to obtain the modified gelatin.

11. The process of preparing the ink formulation as claimed in claim 7, wherein the tunable gelation time ranges for 1 minute to 120 minutes.
